(19) United States
(12) Patent Application Publication
(10) Pub. No.: US 2026/0294896 A1
(43) Pub. Date: Oct. 1, 2026

(54) TRIMETAZIDINE SOLID PHASES

(71) Applicant: LES LABORATOIRES SERVIER, SURESNES CEDEX (FR)

(72) Inventors: Julien MAHIEUX, Ormes (FR); Frédéric VILLARD, Orléans (FR)

(21) Appl. No.: 19/476,391

(22) PCT Filed: Apr. 19, 2024
(86) PCT No.: PCT/EP2024/060657

§ 371 Date: Oct. 17, 2025

(30) Foreign Application Priority Data
Apr. 20, 2023 (EP) 23315089.5

(51) Int. Cl. A61K 31/495; A61K 9/10; A61K 9/16; A61K 9/19; A61K 9/20; A61K 9/48; A61K 31/138; A61K 47/69

(52) U.S. Cl. CPC A61K 31/495; A61K 9/10; A61K 9/1652; A61K 9/19; A61K 9/2054; A61K 9/2059; A61K 9/4866; A61K 31/138; A61K 47/6921

(57) ABSTRACT

The present invention relates to novel trimetazidine solid phases, leading to a reduced formation of trimetazidine nitrosamine in presence of nitrites.

The present invention relates to novel trimetazidine solid phases.

Trimetazidine, or 1-(2,3,4-trimethoxybenzyl)piperazine (formula (I)), is a compound which, by maintaining the energy metabolism of a cell exposed to hypoxia or ischaemia, avoids the collapse of the intracellular level of adenosine triphosphate (ATP). It thus ensures functioning of the ion pumps and sodium-potassium transmembrane flows and maintains cellular homeostasis.

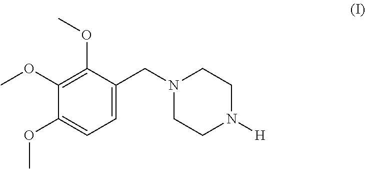 [SMILES: COc1ccc(CN2CCNCC2)c(OC)c1OC]

Trimetazidine, in its free base form, is a liquid at room temperature.

The trimetazidine dihydrochloride salt is disclosed in GB929252 and is currently used therapeutically for the prophylactic treatment of angina pectoris crisis, in chorioretinal attacks and for the treatment of vertigo of vascular origin (Meniere's vertigo, tinnitus).

The patent applications CN110105307, CN110183398 and CN110054599 respectively disclose the synthesis of trimetazidine hemioxalate, oxalate and dioxalate salts, in order to overcome flowability, poor tableting property and hygroscopicity issues of the trimetazidine dihydrochloride salt.

Trimetazidine nitrosamine of formula (II) is an impurity which may be formed during the process of synthesis of trimetazidine or trimetazidine salts or during the formulation process of such salts, in presence of nitrites coming from water and/or excipients.

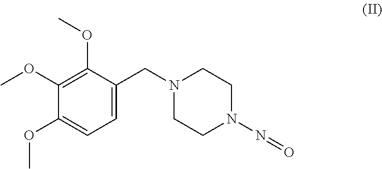 [SMILES: COc1ccc(CN2CCN(N=O)CC2)c(OC)c1OC]

The Applicant has now found that the novel trimetazidine solid phases of the present invention allow to reduce the formation of trimetazidine nitrosamine in presence of nitrites.

In a first embodiment, the present invention relates to trimetazidine solid dispersions.

As used herein, the term “trimetazidine solid dispersion” refers to a dispersion of trimetazidine in its free base, liquid form, in a non-acidic, pharmaceutically acceptable solid matrix compound.

Among the non-acidic pharmaceutically acceptable matrix compounds, there may be mentioned more especially non-acidic pharmaceutically acceptable polymers, preferably cellulose derivatives such as cellulose, pregelatinized starch, cellulose ethers such as hydroxypropylcellulose, hydroxyethylcellulose, hydroxymethylcellulose, methylcellulose or hydroxypropyl methylcellulose.

The preferred non-acidic pharmaceutically acceptable matrix compounds are hydroxypropylmethylcellulose (HPMC), hydroxyethylcellulose and pregelatinized starch, more preferably hydroxypropylmethylcellulose (HPMC).

The weight ratio between trimetazidine and the non-acidic pharmaceutically acceptable matrix compound is preferably about 50/50 or below, more preferably between about 50/50 and about 10/90, for example between about 40/60 and about 20/80, more preferably about 20/80.

The trimetazidine solid dispersions may be prepared by addition of trimetazidine to the matrix compound in solution, followed by freeze-drying of the so-obtained mixture. The trimetazidine solid dispersions are obtained in the form of an amorphous powder.

In another embodiment, the present invention relates to trimetazidine complexes with theophylline.

As used herein, the term “complex” refers to a solid made up of two chemical entities in a defined stoichiometric ratio, linked by weak interactions, typically hydrogen bonding or other non-covalent and non-ionic interactions. In the present invention, the two chemical entities are trimetazidine and theophylline.

The trimetazidine complexes with theophylline may be prepared by stirring trimetazidine and theophylline in a solvent, followed by a slow rise in temperature to the heating temperature, for example about 50° C., maintenance at said temperature for 2 hours, then return to room temperature, additional stirring and finally filtration and drying of the powder thus obtained.

Among the solvents which may be used for the preparation of the trimetazidine complex with theophylline, there may be mentioned acetonitrile, methanol, ethanol, toluene, tetrahydrofuran, ethyl acetate, methyl tert-butyl ether, water or n-heptane.

The present invention also relates to pharmaceutical compositions comprising the trimetazidine complex with theophylline or trimetazidine solid dispersions of the present invention, in combination with one or more inert, non-toxic, pharmaceutically acceptable excipients or carriers.

The useful dosage varies according to the age and weight of the patient, the administration route, the nature and severity of the disorder and any associated treatments, and ranges from 15 mg to 200 mg per day in one or more administrations.

Among the pharmaceutical compositions according to the invention there may be mentioned more especially those that are suitable for oral, parenteral (intravenous, intramuscular or subcutaneous), per- or trans-cutaneous, nasal, rectal, perlingual, ocular or respiratory administration, and especially tablets or dragées, sublingual tablets, gelatin capsules, capsules, minigranules, suppositories, creams, ointments, dermal gels, injectable or drinkable preparations, aerosols, and eye or nasal drops.

The pharmaceutical compositions may be in the form of immediate release or prolonged release compositions.

According to one aspect of the present invention, the pharmaceutical composition is a tablet for immediate release oral administration.

According to another aspect of the present invention, the pharmaceutical composition is a matrix tablet for prolonged release oral administration.

According to another aspect of the present invention, the pharmaceutical composition is in the form of coated minigranules in capsules, for oral, once-a-day administration.

In addition to the trimetazidine solid phase of the present invention, the tablets according to the invention comprise one or more excipients or carriers, such as diluents, retardants, lubricants, binders, disintegrators, absorbents, plasticizers, colourants and sweeteners.

There may be mentioned as examples of excipients or carriers:
for the diluents: lactose, dextrose, sucrose, mannitol, sorbitol, glycerol, calcium hydrogen phosphate dihydrate, calcium carbonate, cellulose, cellulose ethers such as hydroxypropylcellulose, hydroxyethylcellulose, hydroxymethylcellulose, methylcellulose or hydroxypropyl methylcellulose,
for the controlled-release agent: ethylcellulose, ethylcellulose derivatives such as cellulose acetate, cellulose acetate butyrate, cellulose acetate propionate, cellulose acetate phthalate, hydroxypropyl methylcellulose acetate succinate and/or polymethacrylates,
for the lubricants: silica, talc, stearic acid and its magnesium and calcium salts, polyethylene glycol, glycerol behenate or sodium benzoate,
for the binders: aluminium and magnesium silicate, starch, gelatin, tragacanth, methylcellulose, sodium carboxymethylcellulose and polyvinylpyrrolidone,
for the plasticizers: acetyl tributyl citrate, glycerol triacetate, acetyl triethyl citrate, acetyl ethyl citrate, diethyl sebacate, dibutyl sebacate, ethyl phthalate, dibutyl phthalate, polyethylene glycol, glycerol and/or propylene glycol,
for the disintegrants: agar, alginic acid and its sodium salt, effervescent mixtures.

The percentage of active ingredient of formula (I) in the tablet is preferably between 5% and 50% by weight.

According to one aspect of the present invention, the trimetazidine solid phase according to the present invention is administered in association with one or more additional active ingredients. The administration in association may be in the form of a simultaneous or successive co-administration of two or more separate pharmaceutical compositions each containing one of the active ingredients (free association), or in the form of the administration of a fixed-dose combination including two or more active ingredients combined in a single dosage form.

Among the additional active ingredients, there may be mentioned more especially betablockers. According to one aspect of the present invention, the trimetazidine solid phase of the present invention is administered in association with a betablocker in the form of a fixed-dose combination.

Among the betablockers, there may be mentioned more especially metoprolol and bisoprolol. According to a preferred aspect of the present invention, the fixed-dose combination of the trimetazidine solid phase and bisoprolol is a capsule containing the trimetazidine solid phase minigranules in an amount which is equivalent to 80 mg trimetazidine dihydrochloride minigranules, and 5 or 10 mg bisoprolol hemifumarate pellets.

According to another preferred aspect of the present invention, the fixed-dose combination of the trimetazidine solid phase and metoprolol is a capsule containing the trimetazidine solid phase minigranules in an amount which is equivalent to 80 mg trimetazidine dihydrochloride minigranules, and 47.5 or 95 mg metoprolol succinate pellets.

A formulation of bisoprolol hemifumarate pellets is already disclosed in Example 4a of WO2016/071631.

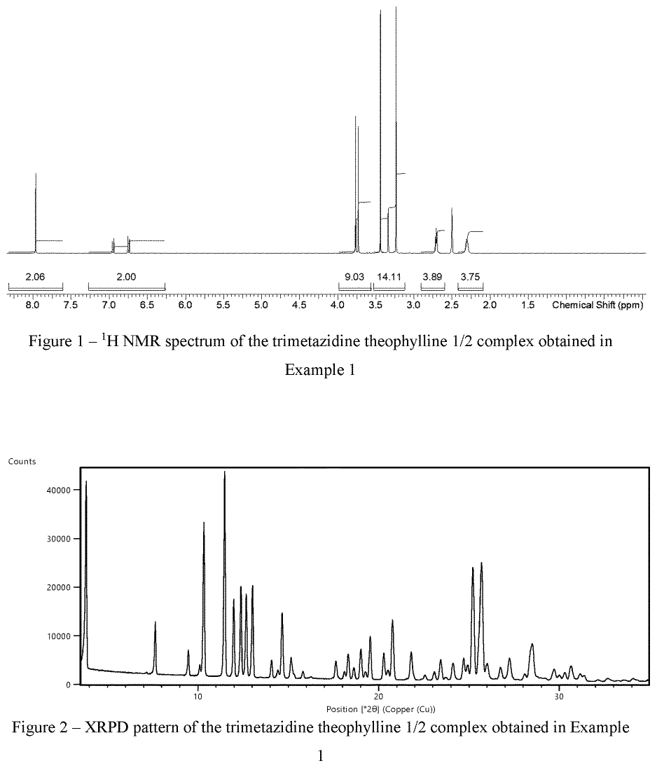

FIG. 1 is the ¹H NMR spectrum of the trimetazidine theophylline ½ complex obtained in Example 1.

FIG. 2 is the XRPD pattern of the trimetazidine theophylline ½ complex obtained in Example 1.

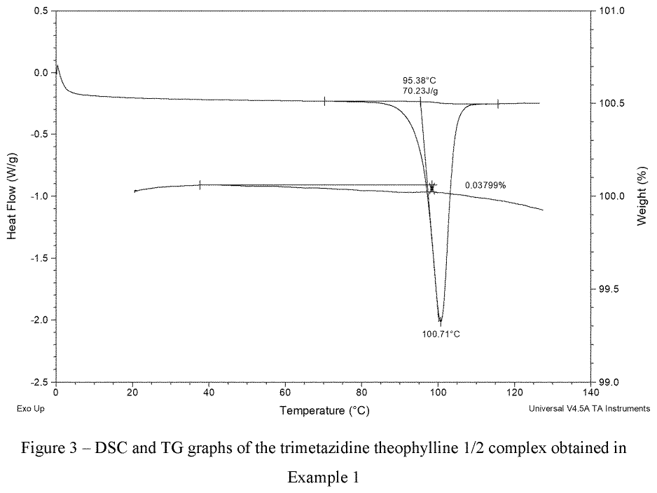

FIG. 3 depicts the DSC and TG graphs of the trimetazidine theophylline ½ complex obtained in Example 1.

### ABBREVIATIONS

DSC Differential Scanning Calorimetry
eq. molar equivalent
HPMC HydroxyPropylMethylCellulose
NMR Nuclear Magnetic Resonance
TG Thermal gravimetry
TMZ Trimetazidine
XRPD X-Ray Powder Diffraction

### EXPERIMENTAL PART

The XRPD pattern was recorded from 3.5 °2θ to 35°2θ using an X-ray diffractometer operating in the transmission mode with CuKa radiation (l=1.5418 Å) at 45 kV and 40 mA and with a 0.013 °2θ step size for 10 minutes.

Only the peaks with a relative intensity higher than 10% have been mentioned in the XRPD table.

The relative intensity may vary in case of preferential orientation.

The chemical shifts, in ppm, are given with respect to tetramethylsilane (TMS), using partially deuterated dimethylsulfoxyde or partially deuterated methanol as internal standard.

In solution in partially deuterated dimethylsulfoxyde, the resonance at 2.5 ppm on the 1D 1H NMR spectrum is due to partially deuterated dimethylsulfoxyde.

The DSC profile was obtained after equilibration step at 0° C. followed by heating step up to 130° C. at 10° C./min. The TG profile was obtained by heating from 25° C. up to 130° C. at 10° C./min.

### Example 1: Trimetazidine Theophylline ½ Complex

10.16 g of trimetazidine was placed in 200 mL of n-Heptane and then 6.98 g of theophylline was added in the solution. The mixture was placed under stirring for 25 minutes at ambient temperature, heated up to 50° C. at 1° C./min followed by isothermal step at 50° C. for 2 hours and finally cooled down to 20° C. at 0.1° C./min followed by isothermal step for 12 hours. The suspension was filtered, and the powder was dried at 40° C. under vacuum overnight. The obtained powder corresponds to an anhydrous crystalline powder with the ½ stoichiometry (Trimetazidine/Theophylline) and a melting point at approximately 95° C.

1H NMR: 9.61 ppm (1H, b), 7.97 ppm (2H, s); 6.95 and 6.75 ppm (2H, d, J₁₂₋₁₃=8.50 Hz); 3.77 and 3.73 ppm (9H, s); 3.44 ppm (6H, s); 3.34 ppm (2H, s); 3.24 ppm (6H, s); 2.71 ppm (4H, bt); 2.30 ppm (4H, bt)

$$b = broad , s = singulet , t = triplet$$

The corresponding 1H NMR spectrum is in FIG. 1.

XRPD table:

| No. | Pos. [°2θ] | Rel. Int. [%] |
|---|---|---|
| 1 | 3.7985 | 79.99 |
| 2 | 7.6297 | 24.49 |
| 3 | 9.4593 | 12.4 |
| 4 | 10.3205 | 72.85 |
| 5 | 11.4641 | 100 |
| 6 | 11.9711 | 37.67 |
| 7 | 12.3671 | 45.51 |
| 8 | 12.6611 | 40.04 |
| 9 | 13.0052 | 44.87 |
| 10 | 14.6497 | 31.73 |
| 11 | 18.3028 | 13.3 |
| 12 | 19.001 | 15.62 |
| 13 | 19.5134 | 22.53 |
| 14 | 20.2697 | 13.89 |
| 15 | 20.7522 | 30.86 |
| 16 | 21.7879 | 14.25 |
| 17 | 23.4148 | 10.86 |
| 18 | 24.686 | 11.2 |
| 19 | 25.1912 | 60.44 |
| 20 | 25.649 | 54.62 |
| 21 | 27.2195 | 11.55 |
| 22 | 28.3875 | 13.6 |
| 23 | 28.513 | 14.3 |

The corresponding XRPD pattern is in FIG. 2.

The DSC/TG graphs are in FIG. 3.

### Example 2: Trimetazidine Solid Dispersions

### Example 2a. Trimetazidine/Non-Acidic Polymer 20/80 Solid Dispersions

### TMZ/HPMC 20/80:

2.4 g of HPMC were slowly added in 350 mL of water and after complete dissolution, 0.64 g of trimetazidine was added in the solution. The mixture was freeze dried under vacuum at 0.05 mbar and at the end, an amorphous powder was obtained.

Other examples of TMZ/non-acidic polymer 20/80 solid dispersions, including TMZ/hydroxyethylcellulose 20/80, TMZ/methylcellulose 20/80 and TMZ/pregelatinized starch 20/80 solid dispersions are prepared with the same operating conditions as above, replacing HPMC with hydroxyethylcellulose, methylcellulose or pregelatinized starch, respectively.

### Example 2b. Trimetazidine/Non-Acidic Polymer Solid Dispersions with Other Ratios

### TMZ/HPMC 50/50:

2.0 g of HPMC were slowly added in 350 mL of water and after complete dissolution, 2.0 g of trimetazidine was added in the solution. The mixture was freeze dried under vacuum at 0.05 mbar and at the end, an amorphous powder was obtained.

Other examples of trimetazidine solid dispersions, including TMZ/HPMC 40/60 and TMZ/HPMC 30/70 solid dispersions, are prepared with the same operating conditions as above by adapting the trimetazidine and HPMC amounts.

### Example 3: Comparative Experiment: Trimetazidine Nitrosamine Formation

In order to model the formation of trimetazidine nitrosamine at the solid state, trimetazidine solid phases (salts, complex, solid dispersions) products containing excipients with high nitrite content were prepared. The sodium nitrite mimics the presence of nitrites in drug product.

Preparation of Excipients with High Nitrite Content:

The first step consists to prepare 100 mL of a sodium nitrite solution at 1 mg/mL.

a) Pregelatinized Starch with High Nitrite Content:
4 g of pregelatinized starch were placed in 500 mL of water and after complete dissolution, 3 mL of the sodium nitrite solution were added. The solution was placed at −40° C. for freezing and then freeze-drying process was performed to obtain a powder.
   b) Hydroxyethylcellulose with High Nitrite Content:
2.8 g of hydroxyethylcellulose were placed in 500 mL of water and after complete dissolution, 2.1 mL of the sodium nitrite solution were added. The solution was placed at −40° C. for freezing and then freeze-drying process was performed to obtain a powder.
   c) HPMC with High Nitrite Content:
4.8 g of HPMC were placed in 500 mL of water and after complete dissolution, 3.6 mL of the sodium nitrite solution were added. The solution was placed at −40° C. for freezing and then freeze-drying process was performed to obtain a powder.

### Preparation of Physical Mixtures Between Trimetazidine Salts or Cocrystal and High Nitrite Content Excipients:

Physical mixtures between salts or cocrystal and high nitrite content excipients were prepared in a 25 mL glass vial by mixing of the two powders with a spatula.

| Trimetazidine | Trimetazidine | High nitrite content |
|---|---|---|
| solid phase | (mg) | excipient (mg) |
| Theophylline cocrystal | 235 | 400 |
| Dichloride salt | 127 | 400 |
| Monochloride salt | 114 | 400 |
| Dioxalate salt | 168 | 400 |
| Oxalate salt | 134 | 400 |
| Hemioxalate salt | 117 | 400 |

Preparation of Trimetazidine Solid Dispersions with High Nitrite Content:
a) TMZ/Pregelatinized Starch 20/80 Solid Dispersions with High Nitrite Content:
400 mg of pregelatinized starch were placed in 500 mL of water and after complete dissolution, 100 mg of trimetazidine base and 0.3 mL of the sodium nitrite solution were added. The solution was placed at −40° C. for freezing and then freeze-drying process was performed to obtain a powder.
   b) TMZ/Hydroxyethylcellulose 20/80 Solid Dispersions with High Nitrite Content:
400 mg of hydroxyethylcellulose were placed in 500 mL of water and after complete dissolution, 100 mg of trimetazidine base and 0.3 mL of the sodium nitrite solution were added. The solution was placed at −40° C. for freezing and then freeze-drying process was performed to obtain a powder.
   c) TMZ/HPMC 20/80 Solid Dispersions with High Nitrite Content:
400 mg of HPMC were placed in 500 mL of water and after complete dissolution, 100 mg of trimetazidine base and 0.3 mL of the sodium nitrite solution were added. The solution was placed at −40° C. for freezing and then freeze-drying process was performed to obtain a powder.

| TMZ/excipient ratio | TMZ solid dispersion (mg) |
|---|---|
| TMZ/HPMC 20/80 | 250 |
| TMZ/Hydroxyethylcellulose 20/80 | 250 |
| TMZ/Pregelatinized starch 20/80 | 250 |

The trimetazidine solid dispersions with high nitrite content and physical mixtures between trimetazidine salts or cocrystal and high nitrite content excipients were placed at 40° C./75% RH in open glass vial for 2 weeks.

### Results: Trimetazidine Nitrosamine Dosage.

The sodium nitrite added in excipients allows in theory to form approximately 10 000 ppm of trimetazidine nitrosamine if the formation was total.

The trimetazidine nitrosamine dosage was performed by LC/MS. The method allows to precisely quantify trimetazidine nitrosamine up to 300 ppm. Above the values are out of the linearity of the method and the values can just be used for tendency and not to quantify the percentage of trimetazidine nitrosamine formation. They were classified as follow:
A: trimetazidine nitrosamine content between 300 and 500 ppm
B: trimetazidine nitrosamine content between 500 and 1000 ppm
C: trimetazidine nitrosamine content between 1000 and 2000 ppm
D: trimetazidine nitrosamine content between 2000 and 3000 ppm
E: trimetazidine nitrosamine content >3000 ppm

### a) Products Containing High Nitrite Content Pregelatinized Starch:

| | Trimetazidine | Trimetazidine nitrosamine |
|---|---|---|
| | nitrosamine | content after 2 weeks at |
| | initial | 40° C./75% RH |
| TMZ solid phase | content | in open glass vial |
| Theophylline cocrystal | 6.6 | A |
| Dichloride salt | 0.2 | E |
| Monochloride salt | 0.8 | C |
| Dioxalate salt | 0.5 | D |
| Oxalate salt | 0.6 | C |
| Hemioxalate salt | 0.2 | C |
| TMZ/Pregelatinized starch 20/80 | 0.2 | A |
| solid dispersion | | |

b) Products containing high nitrite content hydroxyethylcellulose:

| | Trimetazidine | Trimetazidine nitrosamine |
|---|---|---|
| | nitrosamine | content after 2 weeks at |
| | initial | 40° C./75% RH in |
| TMZ phase | content | open glass vial |
| Theophylline cocrystal | 6.6 | 144 ppm |
| Dichloride salt | 0.2 | E |
| Monochloride salt | 0.8 | D |
| Dioxalate salt | 0.5 | D |
| Oxalate salt | 0.6 | D |
| Hemioxalate salt | 0.2 | C |
| TMZ/ | 0.2 | 121 ppm |
| Hydroxyethylcellulose | | |
| 20/80 | | |
| solid dispersion | | |

### c) Products Containing High Nitrite Content HPMC:

| | Trimetazidine | Trimetazidine nitrosamine |
|---|---|---|
| | nitrosamine | content after 2 weeks at |
| | initial | 40° C./75% RH in |
| TMZ phase | content | open glass vial |
| Theophylline cocrystal | 6.6 | 111 ppm |
| Dichloride salt | 0.2 | D |
| Monochloride salt | 0.8 | C |
| Dioxalate salt | 0.5 | A |
| Oxalate salt | 0.6 | A |
| Hemioxalate salt | 0.2 | 209 ppm |
| TMZ/HPMC 20/80 solid | 0.2 | 121 ppm |
| dispersion | | |

The above results show that the trimetazidine solid phases of the present invention allow to reduce the formation of trimetazidine nitrosamine in presence of nitrites, compared to the trimetazidine salts of the prior art.

### Example 4: Immediate Release Formulation—Corresponding to a Formulation Containing 20 mg Trimetazidine Dihydrochloride

Trimetazidine solid phase . . . 36.94 or 78.50 mg (see the table below)
Maize starch . . . 26 mg
Mannitol . . . 34 mg
Polyvidone . . . 4 mg
Magnesium stearate . . . 1 mg
Talc . . . 5 mg

Equivalence table

| | | | Amount of trimetazidine |
|---|---|---|---|
| | | | solid phase in |
| | Trimetazidine | M | the IR tablet |
| Compound | solid phase | (g/mol) | formulation (mg) |
| Example 1 | 1/2 complex with | 626.67 | 36.94 |
| | theophylline | | |
| Example 2a | TMZ/non-acidic polymer | / | 78.50 |
| | 20/80 solid dispersion | | |

Other immediate release formulations, containing TMZ/non-acidic matrix compound solid dispersions with other ratios, are prepared by adjusting the amount of the trimetazidine solid dispersion.

### Example 5: MR Matrix Tablet—Corresponding to the Matrix MR Tablet Containing 35 mg Trimetazidine Dihydrochloride

Trimetazidine solid phase . . . 64.65 or 137.40 mg (see the table below)
Hydroxypropyl methylcellulose . . . 74.0 mg
Povidone . . . 8.7 mg
Calcium hydrogen phosphate dihydrate . . . 80.9 mg
Magnesium stearate . . . 1.0 mg
Anhydrous colloidal silica . . . 0.4 mg

Equivalence table

| | | | Amount of trimetazidine |
|---|---|---|---|
| | | | solid phase in |
| | Trimetazidine solid | M | the MR matrix tablet |
| Compound | phase | (g/mol) | formulation (mg) |
| Example 1 | 1/2 complex with | 626.67 | 64.65 |
| | theophylline | | |
| Example 2a | TMZ/non-acidic | | 137.40 |
| | polymer 20/80 solid | | |
| | dispersion | | |

Other MR matrix tablets, containing TMZ/non-acidic matrix compound solid dispersions with other ratios, are prepared by adjusting the amount of the trimetazidine solid dispersion.

### Example 6: OD Formulation—Corresponding to the OD Formulation Containing 80 mg Trimetazidine Dihydrochloride

Trimetazidine solid phase . . . 147.77 or 266.85 mg (see the table below)
Neutral sucrose/maize starch minigranules . . . 36.67 mg
Hydroxypropyl methylcellulose . . . 6.40 mg
Acetyl tributyl citrate . . . 1.20 mg
Ethylcellulose . . . 8.00 mg
Talc . . . 12.00 mg
Magnesium stearate . . . 0.43 mg

Equivalence table

| | | | Amount of trimetazidine |
|---|---|---|---|
| | Trimetazidine solid | M | solid phase in |
| Compound | phase | (g/mol) | the OD formulation (mg) |
| Example 1 | 1/2 complex with | 626.67 | 147.77 |
| | theophylline | | |
| Example 2a | TMZ/non-acidic | | 266.85 |
| | polymer 20/80 solid | | |
| | dispersion | | |

Other OD formulations, containing TMZ/non-acidic matrix compound solid dispersions with other ratios, are prepared by adjusting the amount of the trimetazidine solid dispersion.

### Example 7: OD Formulation—Combination with Bisoprolol Hemifumarate

The trimetazidine solid phase minigranules obtained in Example 6 and bisoprolol hemifumarate pellets containing 5 mg bisoprolol hemifumarate are mixed together and encapsulated.

### Example 8: OD Formulation—Combination with Metoprolol Succinate

The trimetazidine solid phase minigranules obtained in Example 6 and metoprolol succinate pellets containing either 47.5 mg, or 95 mg metoprolol succinate are mixed together and encapsulated.

1-8. (canceled)

9. A trimetazidine solid phase, selected from trimetazidine solid dispersions in a non-acidic pharmaceutically acceptable matrix compound, and trimetazidine complexes with theophylline, wherein a trimetazidine solid dispersion is a dispersion of trimetazidine in its free base, liquid form, in a non-acidic, pharmaceutically acceptable solid matrix compound.

10. The trimetazidine solid dispersion according to claim 9, wherein the non-acidic pharmaceutically acceptable matrix compound is a polymer.

11. The trimetazidine solid dispersion according to claim 10, wherein the non-acidic pharmaceutically acceptable matrix compound is a pregelatinized starch or cellulose derivative.

12. The trimetazidine solid dispersion according to claim 10, wherein the non-acidic pharmaceutically acceptable matrix compound is hydroxypropylmethylcellulose or hydroxyethyl cellulose.

13. The trimetazidine solid dispersion according to claim 9, wherein the weight ratio between trimetazidine and the non-acidic pharmaceutically acceptable matrix compound is about 50/50 or below.

14. The trimetazidine solid dispersion according to claim 13, wherein the weight ratio between trimetazidine and the non-acidic pharmaceutically acceptable matrix compound is between about 50/50 and about 10/90.

15. The trimetazidine solid dispersion according to claim 14, wherein the weight ratio between trimetazidine and the non-acidic pharmaceutically acceptable matrix compound is about 20/80.

16. A pharmaceutical composition comprising the trimetazidine solid phase according to claim 9, in combination with one or more inert, non-toxic, pharmaceutically acceptable excipients or carriers.

17. The pharmaceutical composition according to claim 16, which is in the form of an immediate-release oral tablet, in the form of a prolonged release oral matric tablet or in the form of coated minigranules in capsules for oral, once-a-day administration.

18. The pharmaceutical composition according to claim 16, further comprising a betablocker.

19. The pharmaceutical composition according to claim 18, wherein the betablocker is metoprolol or bisoprolol.

20. A method for prophylactic treatment of angina pectoris, in the course of chorioretinal disorders or for the treatment of vertigo of vascular origin, in a subject in need thereof, comprising administration of the trimetazidine solid phase according to claim 9.

21. A method for prophylactic treatment of angina pectoris, in the course of chorioretinal disorders or for the treatment of vertigo of vascular origin, in a subject in need thereof, comprising administration of the pharmaceutical composition according to claim 17.

22. A method for prophylactic treatment of angina pectoris, in the course of chorioretinal disorders or for the treatment of vertigo of vascular origin, in a subject in need thereof, comprising administration of the pharmaceutical composition according to claim 18.
